(19) United States
(12) Patent Application Publication
(10) Pub. No.: US 2011/0144624 A1
(43) Pub. Date: Jun. 16, 2011

(54) SLEEVE APPLICATOR TOOL

(75) Inventor: Christine Glaister, Seattle, WA (US)

(73) Assignee: HEALIONICS CORPORATION, Seattle, WA (US)

(21) Appl. No.: 12/965,722

(22) Filed: Dec. 10, 2010

Related U.S. Application Data
(60) Provisional application No. 61/285,515, filed on Dec. 10, 2009.

(51) Int. Cl. A61M 25/00; B23P 19/04

(52) U.S. Cl. 604/524; 29/235

(57) ABSTRACT

Described herein is an applicator tool for sleeving an elongated device, the tool including a hollow, continuous base section, at least two arm members extending laterally from the base section, the arm members and the base section defining an axial passage extending therethrough, and an elastomeric sleeve positioned over a portion of an outer peripheral surface of the arm members and axially aligned with the axial passage, wherein each arm member is radially flexible such that the axial passage can be radially compressed or expanded, and wherein the axial passage allows for a movable positioning of the elongated device.

### BACKGROUND

1. Technical Field

This invention can be used in any field where it is desired to conveniently slide and position a flexible sleeve over a generally tubular or an elongated item. It is particularly directed to applying an elastomeric sleeve or cuff over a medical catheter so as to allow custom location fitting for individual patients.

2. Description of the Related Art

Positioning a flexible sleeve over a tubular or other generally elongate item can be very difficult. The flexible sleeve can stretch, wrinkle and roll up on itself, making it hard to manipulate and locate as desired. The process is especially difficult if the sleeve is intended to be radially stretched and then released so as to create a tight fit onto the tubular item. In a medical setting catheters and other percutaneous devices such as feeding tubes may often be fitted with sleeves or cuffs at the patient's skin exit point or at other, more subcutaneous locations. These cuffs are often constructed of textured or porous polymer materials so as to encourage tissue ingrowth and thus firmly anchor the catheter against unwanted movement. The most common arrangement has the catheter supplied with the cuff (or cuffs), located and adhered at a fixed position along the tube. An example is the MIC Jejunostomy Feeding Tube, Model 03010-14, made by Kimberly Clark Health Care.

To allow adaptation to the body habitus of specific patients a catheter with a cuff in a predetermined location along the tube might be altered by removing part of the distal end. However, this end is often provided with specific features such as side ports, to facilitate the intended purpose of the catheter and cannot be altered. This makes the provision of a cuff that can be conveniently applied and positioned during catheter placement a desirable objective. Such cuffs may be constructed from elastomeric materials to an internal dimension that is less than that of the outer dimension of the catheter. Thus when applied to the catheter the cuff is in an elastically tensioned state and firmly retained in the desired position. In order to achieve this end it is necessary to provide a means to radially stretch the cuff, position it on the catheter and then release the applicator means. Preferably the applicator should be easily sterilized and disposable.

A technically similar requirement is found in the electronics industry where the need exists to apply sections of elastic tubing over wire splices. Tube expanding tools such as taught in U.S. Pat. No. 5,014,407 can be used to stretch open such tube sections and position them over an inner device, but such tools have multiple shortcomings and disadvantages in a medical setting. These include the use of moveable pins inserted into the sleeve for stretching. These pins can create stress concentrations that damage the sleeve and may also deform under the force of stretching the sleeve thereby closing down on the item being sleeved and preventing further movement. In a medical setting these mechanically complex devices cannot economically be made disposable and thus present difficulties in repeatedly securing and maintaining sterility. A further disadvantage is that specific tools are useable only over a limited range of elastic tubing sizes and provision of interchangeable pin sets for different size ranges creates a yet more complex tool, compounding the problems of sterilization, difficulty of use during a medical procedure and cost.

Clearly there is a need for improved sleeving of elongated devices such as catheters, tubes, wires and the like. The presently disclosed invention embodiments address this need and provide other related advantages.

### BRIEF SUMMARY

One embodiment of the invention described herein provides a tool having at least two elongate arms connected at one end by a generally cylindrical section of greater internal dimensions that the outer dimension of the sleeved device and where the arms are generally separated by spaces allowing them to be compressed together into the lumen of the sleeve. After the sleeve is placed over the tool the arms are allowed to relax back and retain the sleeve. The tool and sleeve may most often be provided in this preassembled form for the medical procedure.

Thus, one embodiment provides a tool for sleeving an elongated device, comprising: a hollow, continuous base section; at least two arm members extending laterally from the base section, the arm members and the base section defining an axial passage extending therethrough; and an elastomeric sleeve positioned over a portion of an outer peripheral surface of the arm members and axially aligned with the axial passage, wherein each arm member is radially flexible such that the axial passage can be radially compressed or expanded, and wherein the axial passage allows for a movable positioning of the elongated device.

In various embodiments, the tool has arm members that are compressed and the elastomeric sleeve that has an internal dimension at a relaxed state that is larger than a radial dimension of the portion of the outer peripheral surface of the compressed arm members.

In other embodiments, an internal dimension of the elastomeric sleeve is smaller than an outer dimension of the elongated device. Furthermore, at least one arm member has a notch positioned between the elastomeric sleeve and the base section. The arm members are formed of a material having a friction coefficient of no more than 0.4, or preferably no more than 0.25 (such as PTFE). The tool may have three, four, five, six, seven or eight arms.

In various embodiments, the elongated device is a catheter and the sleeve is a cuff; or the elongated device is a wire and the sleeve is an insulating sleeve, or the elongated device comprises a bundle of cables and the sleeve is an elastic cord. In use, the tool and sleeve together are slid over the elongated device (e.g., catheter), with the tool cylindrical section leading. Most often the fitting will be done starting from the distal end of the catheter. Pulling the tool and sleeve over the catheter then expands the sleeve by just the amount needed for easy positioning. When positioned in the desired location the arms are folded back releasing the sleeve. The tool material, shape and dimensions are chosen so as to provide a semi rigid structure with sufficient strength to resist deformation and retain the pre-fitted sleeve while readily bending when greater force is applied to extract the arms. The cylindrical section is provided with notches or grooves allowing it to be broken and removed along with the arms after sleeve placement.

In a preferred embodiment the tool is constructed of polytetrafluoroethylene (PTFE) to provide a highly biocompatible, low friction surface.

While the tool construction and use is described most fully for applying sleeves to devices of generally circular cross section, it will be understood that the design is readily adaptable to use with sleeves and devices of any cross-section, including but not limited to elliptical and polygonal.

A further embodiment provides a catheter assembly comprising: a catheter having an outer diameter; an applicator for sleeving the catheter, the applicator including: a hollow, continuous base section; at least two arm members extending laterally from the base section, the arm members and the base section defining an axial passage extending therethrough; and an elastomeric sleeve positioned over a portion of an outer peripheral surface of the arm members and axially aligned with the axial passage, wherein each arm member is radially flexible such that the axial passage can be radially compressed or expanded, and the catheter is movably positioned in the axial passage.

In various embodiments, the catheter assembly comprises arm members that are compressed and the elastomeric sleeve that has an internal dimension at a relaxed state that is larger than a radial dimension of the portion of the outer peripheral surface of the compressed arm members.

In other embodiments, an internal dimension of the elastomeric sleeve is smaller than an outer dimension of the elongated device. Furthermore, at least one arm member has a notch positioned between the elastomeric sleeve and the base section. The arm members are formed of a material having a friction coefficient of no more than 0.4, or preferably no more than 0.25 (such as PTFE). The tool may have three, four, five, six, seven or eight arms.

### BRIEF DESCRIPTION OF THE SEVERAL VIEWS OF THE DRAWINGS

FIG. 1 shows an exemplary form of the tool, comprising 4 arms.

FIG. 2A shows the tool arms compressed to allow fitting of the sleeve over the tool.

FIG. 2B is one exemplary arrangement of the compressed arms.

FIG. 2C shows an alternative arrangement of the compressed arms.

FIG. 3A shows the tool arms compressed and ready to receive the sleeve.

FIG. 3B shows the tool arms relaxed and retaining the sleeve.

FIG. 4 shows fitting of the tool and sleeve to a catheter.

FIG. 5A shows an arm flexed.

FIG. 5B shows an arm released.

FIG. 6 shows the arm dimensioning needed to avoid sleeve contact with the sleeved device during positioning.

### DETAILED DESCRIPTION

Certain preferred embodiments of the present invention provide an applicator tool to facilitate the fitting and location of flexible sleeves onto generally tubular or elongated devices. The applicator construction provides for expanding the sleeve to a greater internal size than the outer dimensions of the tubular item, while avoiding stress concentrations in the sleeve and further, for reducing friction against the device, thus allowing ready positioning. The design then further provides for ready removal of the applicator from the positioned sleeve and from the device. The design is particularly adapted to use in a medical environment where the devices are often catheters and where inherently simple, low cost and disposable items are advantageous.

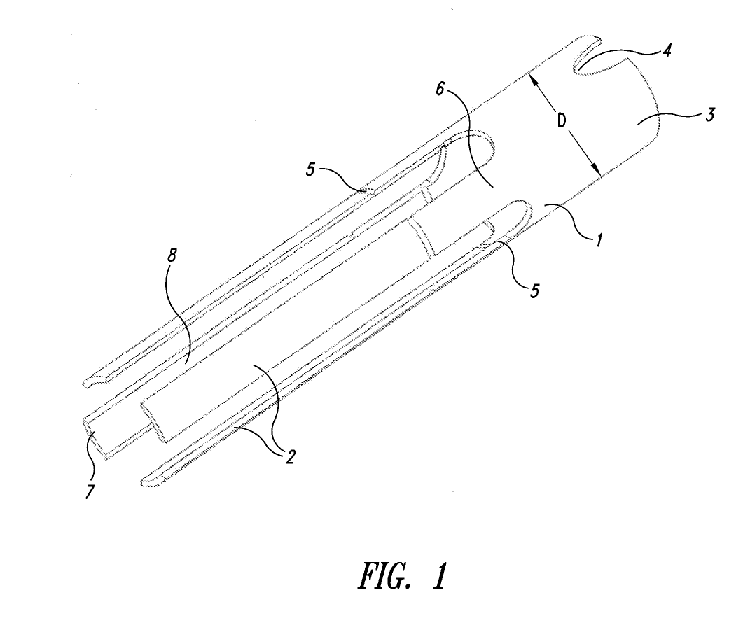

With reference to the figures, an exemplary form of the applicator tool for sleeving an elongated device is shown in FIG. 1. The tool 1 is formed from a single piece of material into two sections, comprising: a) two or more separated arm members (“arms”) 2, optionally provided with one or more notches 5 to control the mechanical flexure and strength properties; and b) a generally cylindrical base section, 3, from which the arms 2 extend laterally, the arm members 2 and the base section 3 defining an axial passage 8 extending therethrough. The base section may often be fitted with a breakaway notch or notches, 4. The arms may often be tapered in width from their proximal end 6 towards the distal end 7. While the base section in FIG. 1 is shown as a circular cylinder it will be appreciated that the base section is not limited to a particular shape so long as it is hollow and can accommodate the elongated device that extends through the axial passage. Thus, many other shapes such as elliptical, square, or polygonal are suitable.

Specific dimensions of the tool are selected for particular applications. It is generally advantageous to make the internal diameter of the tool (e.g., the diameter of the axial passage when the arms are not compressed) about 30% to about 70% greater than the outer diameter of the elongated device to be sleeved. To allow for arm compression and retention of the sleeve the arms may often be made from about 150% to about 250% the length of the sleeve, and the relative width of the arms at their widest section to the spaces between them may be made in the range of about 75% to about 125%, most often about 100%.

As used herein, “compressing” or “compression of” the arms refer to arranging, in any manner, to bring the arms from a relaxed state (or a less compressed state) closer together such that the dimension of the axial passage is reduced radially. Typically, compression causes the distal ends of the arms, which are spaced apart from each other in a relaxed state, to stack into a compacted state. As a result of the compression at the distal ends of the arms, the radial dimensions of the axial passage are reduced on a declining scale along the length of the arms, the reduction at the distal ends being larger than the reduction at the proximal ends of the arms.

Compression of the arms may be done manually or by any number of simple tools as will occur to one versed in elementary mechanics. When compressed the arms may take up a variety of arrangements.

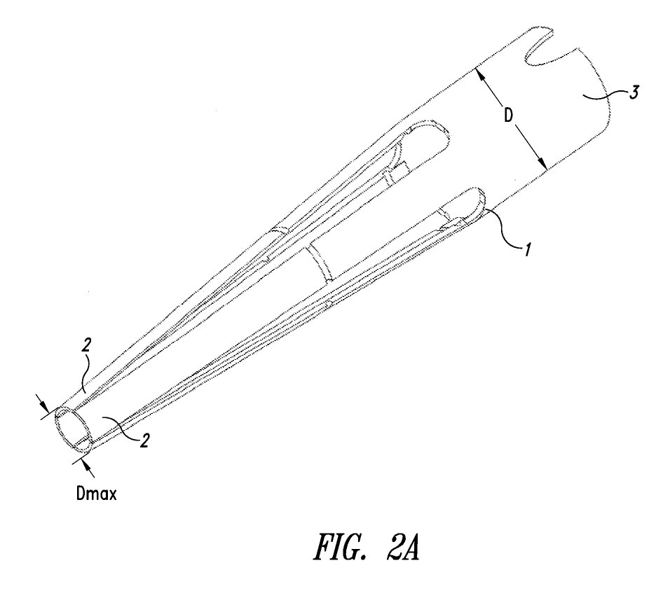

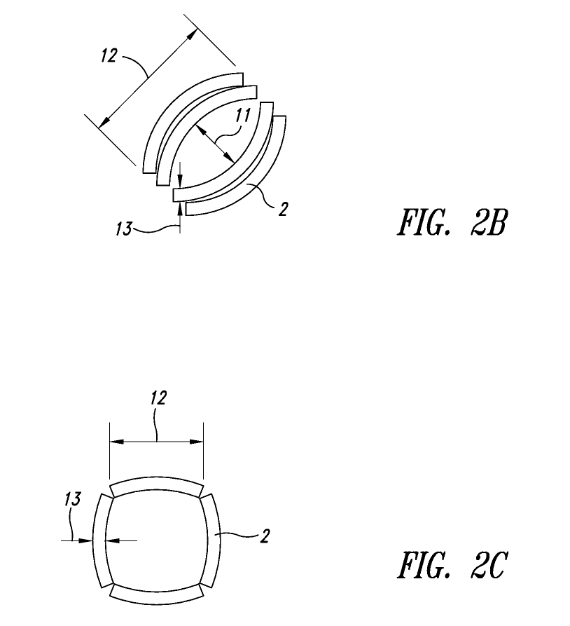

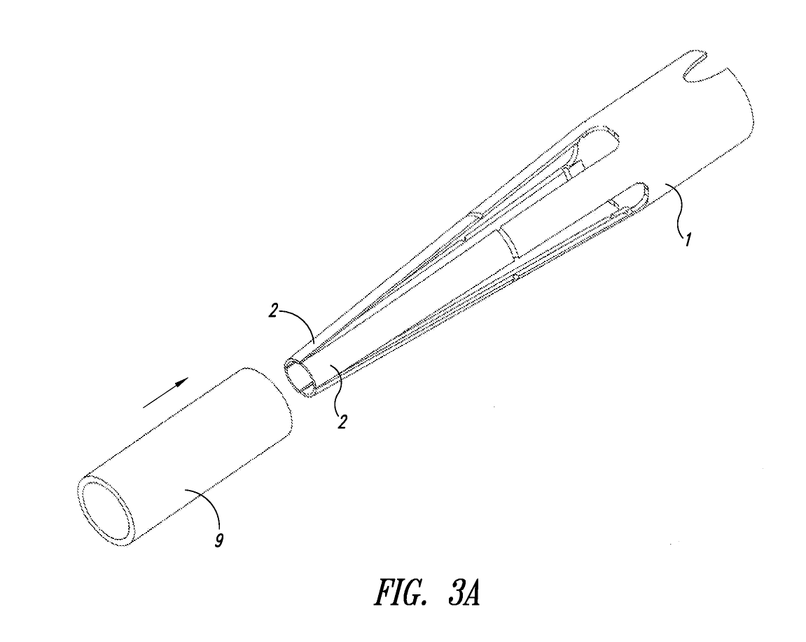

FIG. 2A shows a manner of compression in which the arms are pressed into contact at their respective distal ends 2 without substantial overlap of the arms. As shown, this arrangement allows for about a 2:1 reduction in a maximum dimension across the folded arms (Dmax) relative to the dimension of the base section (D). The dimensions of the compacted state of the arms correlate to the degree of compression, with Dmax being the maximum radial dimension of a compacted state of the arms (as measured on the periphery of the compressed arms), including the space defined by the same. Further compression would lead to a more compacted state of the arms (and a smaller dimension at the distal ends) while the arms are likely distorted or deformed (e.g., causing the curvature of the arms to flatten out). FIGS. 2B and 2C illustrate representative manners in which the arms are compressed, but it should be understood that many other relative numbers and placements of the arms can be easily visualized. In FIG. 2B, for arms of width 12 designated mathematically as “W” and thickness 13 designated as “T”, and formed in this example, from a cylinder 3 of diameter “D”, the largest dimension “Dmax” of the stack is 4T+gap 11, with 11 being the central space between the packed arms. Gap 11, is sufficiently estimated as about twice the height of a chord of width W from the cylinder diameter D; i.e., gap 11 is given by 2(W²/4D) when W is significantly less than D.

In a less tightly packed configuration such as shown in FIG. 2C Dmax is about (√2)W and for the same arm geometry will most often be greater than the maximum dimension observed for the FIG. 2B configuration.

It will be apparent that Dmax should not exceed the sleeve internal diameter when the sleeve is relaxed on the tool. With reference to the FIG. 2C arrangement, most often the arm dimensions W and T should not result in a dimension greater than about 70% of the smallest internal diameter of the applied sleeve and more often about 40% to about 70% of the sleeve internal size.

The tool can have two or more arms. Although the exemplary embodiment shown in the figures has four arms, arrangements of three, five, six, seven or eight arms are possible. The arms can be straight sided, or they can be tapered. While the individual arms may often be made to about the same dimensions, in certain contemplated embodiments they may differ from one another in width or length to suit a particular sleeve design or the geometry of the elongated device.

The tool can be made from a variety of materials. Preferred materials should be semi-rigid, able to hold their shape while maintaining the ability to flex and bend without fracturing.

In various embodiments, a low friction material is used to form the applicator tool. Typically, polymeric materials having coefficients of friction of no more than 0.4, more typically, no more than 0.25, or more typically, no more than 0.15. Preferred polymeric materials include, without limitation, fluorocarbon-based polymers such as polytetrafluoroethylene (PTFE), poly(fluorinated ethylene propylene) (FEP), perfluoroalkoxy (PFA), poly(ethylene-co-tetrafluoroethylene) (ETFE) and poly(ethylene-chlorotrifluoroethylene) (ECTFE).

In addition to having a low friction coefficient, fluorocarbon-based polymers are biocompatible and have high melting points, thus can withstand high-temperature sterilization by, for example, autoclave. These combined characteristics make fluorocarbon-based polymers particularly suited for medical or clinical applications.

The applicator tool further comprises an elastomeric sleeve designed to tightly adhere to the elongated device (e.g., a catheter). The elastomeric sleeve may be made with an internal diameter such that when applied to the more rigid elongated device (e.g., catheter), the sleeve will be expanded by about 20%. On the other hand, the internal dimension of the sleeve at a relaxed state should be larger than a radial dimension of the periphery of the compressed arms of the applicator such that the sleeve is not stretched or under stress prior to being sleeved onto the elongated device.

The applicator tool can be used with sleeves of varying sizes made from a variety of elastomeric materials (i.e., silicone rubber, polyurethane, etc). Most often sleeve materials should be flexible and elastic; however, the degree of flexibility will vary depending on the application and may require variations in the applicator tool dimensions. The exemplary embodiment shown in this disclosure utilizes a sleeve with a circular cross section, but the tool can be used with sleeves with cross sections of any geometry (i.e., polygonal, elliptical, etc).

In certain embodiments, the elongated device is a catheter and the sleeve is a cuff. For example, a sleeve for a 12F catheter would be made with an internal dimension (or a lumen) of about 3.35 mm. To allow easy sleeve placement on the tool, the arms need to fold close enough together so as to readily pass through an aperture of not more than about 3 mm diameter over a length of at least 20 mm. This will be achieved with the arms folded if the arms are separated by gaps as shown in FIG. 1, where the gaps are about the same dimension as the arm width. In this example the arms and gaps are both made about 2.2 mm wide, or W=66% of the sleeve lumen, corresponding to the more generalized calculation described above.

It has been found that, when using PTFE as the tool material, an arm thickness of about 0.4 mm provides a suitable stiffness for the tool use, allowing easy removal after sleeve positioning while not always requiring the use of notches 5 to provide breakaway points. This thickness value also satisfies the minimum requirement as calculated above for providing clearance between the sleeve and the device during sleeve positioning.

As a further illustrative calculation, for W, T and D values of 2.2 mm, 0.4 mm and 6 mm respectively disposed as in FIG. 2B, then Dmax=2.0 mm, and if disposed as in the FIG. 2C arrangement, then Dmax=3.1 mm.

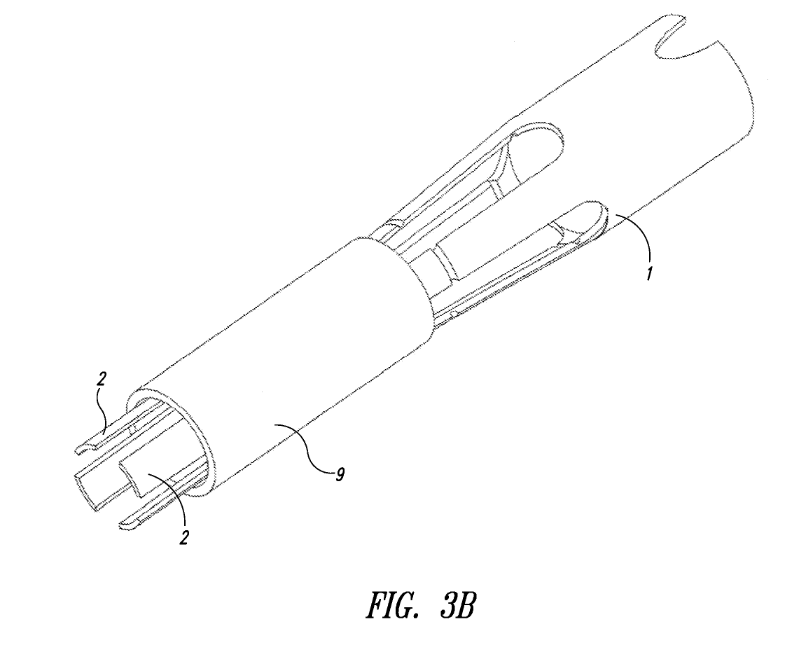

FIG. 3A shows the arms compressed and ready to receive a sleeve 9, and FIG. 3B shows the arms relaxed back to hold the sleeve on the tool. It should be noted that after the sleeve is introduced the arms are not necessarily as fully relaxed as they are prior to any compression (e.g., in the state as shown in FIG. 1). Instead, certain tension remains in order to hold the sleeve taut yet not substantially stretched.

It will readily be seen that, by following the relative proportions, size calculations and examples as specified above, the tool dimensions may be adjusted to any sizing combinations of the sleeved device and the applied sleeve.

The applicator tool can be used on devices of varying sizes that often may be larger or smaller than the sleeve. The device to be sleeved can be rigid, semi-rigid or flexible, and may be made from a variety of materials (e.g., silicone rubber, polyurethane, PEEK, metals). The example shown in this disclosure indicates a device with a circular cross section; however, the tool can be used to apply a flexible/elastomeric sleeve to a generally elongate device of any cross section.

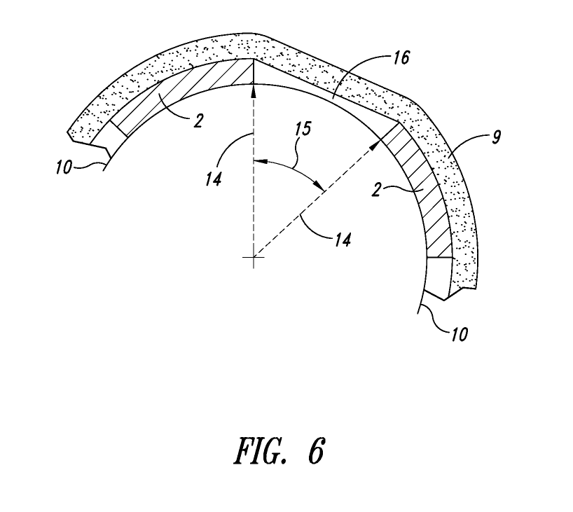

As a further design consideration, in certain embodiments the purpose of applying a sleeve to a device while avoiding friction between the sleeve and device may be achieved when the tool arms are spaced in such a manner as to not allow any part of the sleeve to contact the sleeved device during positioning. The further purpose of minimizing stress on the expanded sleeve will then be realized when the sleeve is separated from the device by a minimal distance to avoid contact. This preferred configuration can be understood by reference to FIG. 6. Shading of the various components shown in FIG. 6 is for clarity of illustration only.

When placed over the target device 10 of outer radius 14, designated as “R” tool arms 2 are pressed against target device 10 by pressure from the expanded sleeve 9 and will assume positions such as those shown in FIG. 6. In the gaps between arms 2 the sleeve will deform to the shortest path and approach the device. If this space between the sleeve and the device approaches zero then both of the design considerations are simultaneously realized.

With an arm 2 thickness 13, designated previously as “T” and a circumferential gap measured by the angle 15, designated as “A” between the edges of arms 2, a geometric calculation will show that the space 16, designated as “t” is given by the equation t=(R+T)cosine(A/2)−R.

In the preferred case where t approaches zero and R is determined by the target device 10 then a trade off may be made between maximum separation of the arms, and the least required thickness, T of the arms by using the equation cosine (A/2)=R/(R+T).

With the further expressed requirement in certain preferred embodiments to permit compression of the arms to less than the relaxed internal diameter of the sleeve, for instance, by including gaps between the arms of about the same width as the arms 2, as specified above, then the value of the angle A will most often be in the range of about π/n radians where n is the number of arms 2. In the exemplary case where n=4 the angle A will be about 45 degrees and the solution for T is about 0.09R. An exemplary 4 mm diameter device would thus require T to be about 0.18 mm minimum thickness. Often tool strength considerations will require a greater thickness.

In a further example with equal arm widths and gaps and n=3, A is about 60 degrees and the solution for T is about 0.12R. For n=5 calculating similarly, T should be at least 0.05R.

As a more concrete example, a tool intended to position 20 mm long sleeves on a 12 French catheter of 4 mm outer diameter (or 2 mm radius “R”) might have an internal diameter of about 6 mm and arms of length preferably between about 30 mm and 40 mm, with the tool overall length of about 40 mm to about 50 mm.

As discussed herein, the applicator tool can be adapted to sleeve a cuff on a catheter. On the other hand, the applicator tool is not limited to uses within a medical setting. Thus, in other embodiments, the elongated device may be a wire or a cable and the sleeve may be an insulating sleeve. In further embodiments, the elongated device may comprise a bundle of cables and the sleeve may comprise an elastic cord. The sleeve serves to keep the cables together and organized.

A further embodiment provides a catheter assembly comprising: a catheter having an outer diameter; and an applicator for sleeving the catheter, the applicator including: a hollow, continuous base section; at least two arm members extending laterally from the base section, the arm members and the base section defining an axial passage extending therethrough; and an elastomeric sleeve positioned over a portion of an outer peripheral surface of the arm members and axially aligned with the axial passage, wherein each arm member is radially flexible such that the axial passage can be radially compressed or expanded, and the catheter is movably positioned in the axial passage.

### EXAMPLE 1

### Method of Tool Manufacture

The applicator tool can be injection molded, stamped out of a tube or cut out of a tube. Other fabrication methods will be apparent to one skilled in the art of polymer device manufacture.

An exemplary method of cutting the applicator tool out of tubing starts by placing the tubing into a fixture specific to the desired tool size and thickness, base length, number of arms, leg length and leg width. The fixture may consist of a central mandrel and an outer sleeve. The central mandrel fits snugly inside the tubing, and the outer sleeve fits snugly around the outside of the tubing. The outer sleeve has cut-outs that correspond to the outline of the arms of the applicator tool. The central mandrel may also have corresponding depressions. These cut outs and depressions are used as guides for cutting the tube into the desired shape. The tube can be cut with a blade, heat knife, laser or other similar means.

### EXAMPLE 2

### Method of Tool Use

To use the applicator tool the following steps are typical:

1. Squeeze arms 2 of the applicator tool together to a packed arrangement as generally shown in FIGS. 2A and 3A. The arms may often lie on top of each other in a stack; they may be bunched together to form a smaller cross section, as shown in FIGS. 2B and 2C respectively or they may assume other packed arrangements provided only that the resultant size is less than about the least inner dimension of the sleeve.

2. Slide sleeve 9 onto the compressed arms 2 as shown in FIG. 3A and release the arms to retain the sleeve as shown in FIG. 3B.

At this stage the sleeve and tool assembly may often be processed and packaged in sterile form using techniques well known in medical device manufacture. Steps 3 through 5 would then first require removal of the assembly from the packaging while observing sterile conditions.

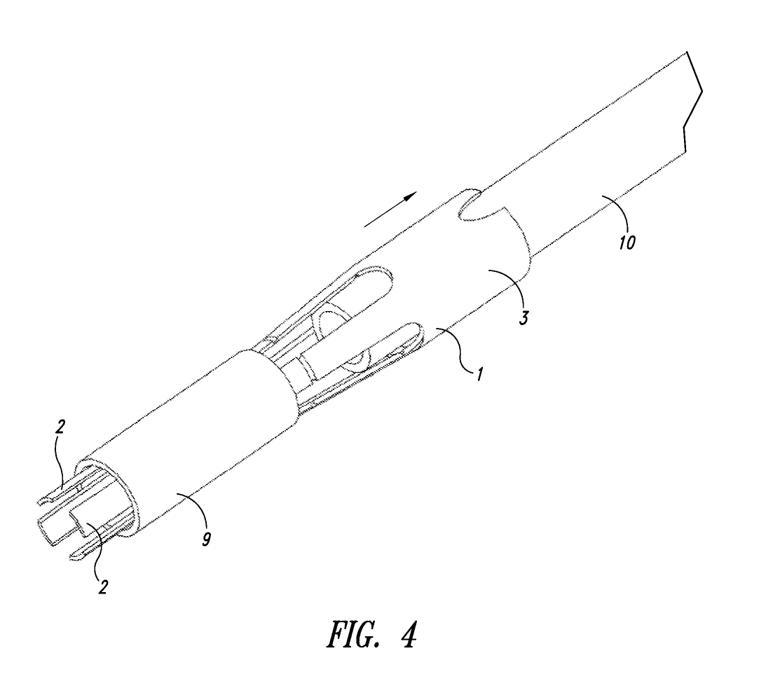

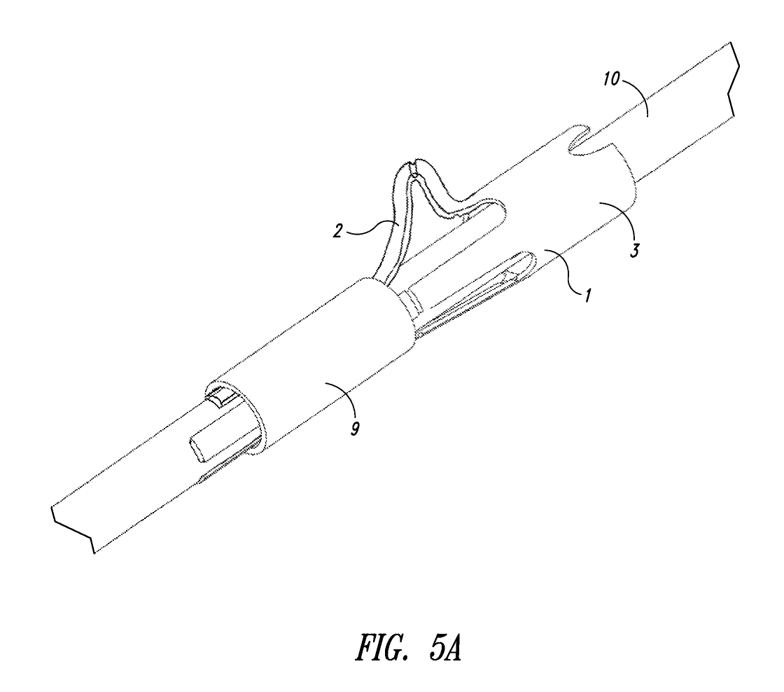

3. Slide the applicator tool 1 onto the device 10 by grasping the base 3 of the applicator tool as shown in FIG. 4 and position the tool and sleeve at the desired location on device 10.

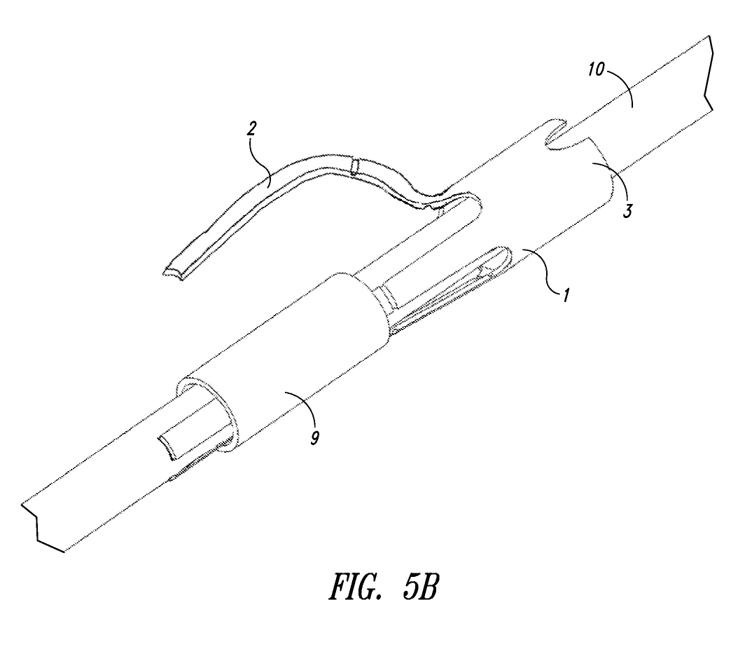

4. Slide the arms of the applicator tool 2 out from under the sleeve 14 usually one at a time as shown in FIGS. 5A and 5B, until all are released.

5. After removing all the arms, the applicator tool many often be removed by sliding off the device, or by tearing section 3 using notch 4, leaving the flexible sleeve 5 in the desired location

It will be understood that this technique may be varied in various particulars related to the exact sizing, shape and materials of the sleeve or device and/or to the method of tool removal, without deviating from the intent and methods of this disclosure. In a medical setting often forceps or a similar device will be used to grasp the preassembled tool and sleeve by the base 3, and slide the assembly along target device 10. While retaining the desired position with the first forceps a second set may be used to manipulate the legs as in FIGS. 5A and 5B, then to break of the applicator tool by twisting 3 at notch 4. It will be understood that individual surgical practice may differ while achieving the same end. The applicator tool is most often treated as a single-use disposable device.

Although the invention has been described with reference to specific embodiments, these descriptions should not be regarded as limiting. Various modifications and alternative embodiments will become apparent to skilled persons on reference to the description. For example the tool could be made by a variety of economical processes other than the one described. While directed in the first instance at applying sleeves to medical devices, the tool design might also be used as an inexpensive disposable device for the application of insulating sleeving to electrical wire connections, especially under conditions where a nonconductive tool is desired.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

I claim:

1. A tool for sleeving an elongated device comprising:
a hollow, continuous base section;
at least two arm members extending laterally from the base section, the arm members and the base section defining an axial passage extending therethrough; and
an elastomeric sleeve positioned over a portion of an outer peripheral surface of the arm members and axially aligned with the axial passage,
wherein each arm member is radially flexible such that the axial passage can be radially compressed or expanded, and wherein the axial passage allows for a movable positioning of the elongated device.

2. The tool of claim 1 wherein the arm members are compressed and the elastomeric sleeve has an internal dimension at a relaxed state that is larger than a radial dimension of the portion of the outer peripheral surface of the compressed arm members.

3. The tool of claim 2 wherein the internal dimension of the elastomeric sleeve is smaller than an outer dimension of the elongated device.

4. The tool of claim 1 wherein at least one arm member has a notch positioned between the elastomeric sleeve and the base section.

5. The tool of claim 1 wherein the arm members are formed of a material having a friction coefficient of no more than 0.4.

6. The tool of claim 1 wherein the arm members are formed of PTFE.

7. The tool of claim 1 comprising three, four, five, six, seven or eight arms.

8. The tool of claim 1 wherein the elongated device is a catheter and the sleeve is a cuff.

9. The tool of claim 1 wherein the elongated device is a wire and the sleeve is an insulating sleeve.

10. The tool of claim 1 wherein the elongated device comprises a bundle of cables and the sleeve is an elastic cord.

11. A catheter assembly comprising:
a catheter having an outer diameter;
an applicator for sleeving the catheter, the applicator including:
(a) a hollow, continuous base section;
(b) at least two arm members extending laterally from the base section, the arm members and the base section defining an axial passage extending therethrough; and
(c) an elastomeric sleeve positioned over a portion of an outer peripheral surface of the arm members and axially aligned with the axial passage,
wherein each arm member is radially flexible such that the axial passage can be radially compressed or expanded, and the catheter is movably positioned in the axial passage.

12. The catheter assembly of claim 1 wherein the arm members are compressed and the elastomeric sleeve has an internal dimension at a relaxed state that is larger than a radial dimension of the portion of the outer peripheral surface of the compressed arm members.

12. The catheter assembly of claim 11 wherein the internal dimension of the elastomeric sleeve is smaller than an outer dimension of the catheter.

13. The catheter assembly of claim 11 wherein at least one arm member has a notch positioned between the elastomeric sleeve and the base section.

14. The catheter assembly of claim 11 wherein the arm members are formed of a material having a friction coefficient of no more than 0.4.

15. The catheter assembly of claim 11 wherein the arm members are formed of PTFE.

16. The catheter assembly of claim 11 wherein the applicator comprises three, four, five, six, seven or eight arms.
